(19) United States
(12) Patent Application Publication
(10) Pub. No.: US 2025/0344947 A1
(43) Pub. Date: Nov. 13, 2025

(54) OPHTHALMOLOGICAL OPTICAL OBSERVATION APPARATUS, METHOD FOR PROVIDING AN OPHTHALMOLOGICAL OPTICAL OBSERVATION APPARATUS WITH A LASER PROTECTION FILTER AND FUNDUS IMAGING SYSTEM

(71) Applicant: CARL ZEISS MEDITEC AG, Jena (DE)

(72) Inventors: Christian BEDER, Aalen (DE); Frank DEUBEL, Oberkochen (DE)

(21) Appl. No.: 19/199,342

(22) Filed: May 5, 2025

(30) Foreign Application Priority Data
May 7, 2024 (DE) 102024112778.3

(51) Int. Cl. A61B 3/12; A61B 3/14

(52) U.S. Cl. CPC A61B 3/12; A61B 3/14; A61B 2576/02

(57) ABSTRACT

The invention relates to an ophthalmological optical observation apparatus for use during a treatment of the eye by means of laser radiation. It comprises a contactless fundus imaging system having at least one optical element and a laser protection filter with a transmission characteristic suitable for blocking the laser radiation used during the treatment of the eye. In this context, at least one optical element of the contactless fundus imaging system is provided with at least one coating that realizes the transmission characteristic and thus forms the laser protection filter.

The present invention relates to an ophthalmological optical observation apparatus for use during a treatment of the eye by means of laser radiation. In addition, the invention relates to a method for providing an ophthalmological optical observation apparatus with a laser protection filter. Moreover, the invention relates to a contactless fundus imaging system.

Ophthalmic surgery without lasers has become inconceivable. They are used both for the treatment of the anterior segment of the eye and for the treatment of the posterior segment of the eye. For example, what is known as an after-cataract may be treated during the treatment of the anterior segment of the eye. The after-cataract is post-operative clouding that follows the insertion of an intraocular lens. Laser treatment can be used to remove this clouding. In the context of treatment of the posterior segment, laser radiation is for example used to re-secure detached regions of the retina to the tissue located behind the retina. To protect the eyes of the physician and, where applicable, of their assistant, use is made of what are known as physician protection filters which for example may be introduced into the beam path of a surgical microscope. For example, such a physician protection filter is described in DE 44 09 506 A1. This radiation protection filter is pivoted-in between a beam splitter that deflects the laser radiation in the direction of the object to be treated and the two main objectives of a surgical microscope. However, such filters require an additional pivoting mechanism below the main objective, rendering the surgical microscope more complex and requiring installation space for the pivoting mechanism.

Moreover, U.S. Pat. No. 5,528,426 A has disclosed a surgical microscope having a beam splitter for output coupling beam paths from the stereoscopic partial beam paths of a stereoscopic main observer beam path. A respective laser protection filter is present at the distal end of the beam splitter for each stereoscopic partial beam path of the main observer beam path.

Vis-à-vis this prior art, a first problem addressed by the present invention is that of making available an ophthalmological optical observation apparatus for use during a treatment of the eye by means of laser radiation and containing a laser protection filter which does not require any additional pivoting mechanism and needs as few elements as possible. In addition, a second problem addressed by the present invention is that of making available a method with which an ophthalmological optical observation apparatus for use during a treatment of the eye by means of laser radiation can be provided with a laser protection filter without requiring an additional pivoting mechanism, wherein the laser protection filter needs as few elements as possible. Moreover, a third problem addressed by the invention is that of making available an advantageous contactless fundus imaging system.

The first problem is solved according to claim 1 by an ophthalmological optical observation apparatus for use during a treatment of the eye by means of laser radiation, the second problem is solved according to claim 6 by a method for providing an ophthalmological optical observation apparatus with a laser protection filter, and the third problem is solved according to claim 11 by a contactless fundus imaging system. The dependent claims contain advantageous configurations of the invention.

According to a first aspect of the invention, an ophthalmological optical observation apparatus for use during the treatment of the eye by means of laser radiation is made available. The ophthalmological optical observation apparatus according to the invention comprises a contactless fundus imaging system having at least one optical element and a laser protection filter with a transmission characteristic suitable for blocking the laser radiation used during the treatment of the eye. According to the invention, at least one optical element of the contactless fundus imaging system is provided with at least one coating that realizes the transmission characteristic and thus forms the laser protection filter.

An optical observation apparatus should be understood to mean an apparatus for observing a tissue region, for example a surgical microscope, a slit lamp, etc., the beam path of which leads to at least one eyepiece and/or to at least one camera.

For example, a transmission characteristic can be represented by a transmission curve, i.e. a curve that represents the transmission as a function of wavelength. In this context, the transmission characteristic of the at least one coating may be realized by a single colored coating, which forms a color filter, or in the form of an interference layer system, which forms an interference filter. The filter effect in a color filter is based on the absorption of the spectral range to be removed, whereas in an interference filter said filter effect is based on the spectral range to be removed being selectively reflected by means of interference. Particularly narrow passbands can be realized with interference filters in particular.

Within the meaning of the invention, laser radiation should be considered blocked if it is attenuated to such an extent that it cannot cause injury to the eye of a user who is observing the treatment region with the optical observation apparatus and/or that it does not lead to the overexposure of an image sensor.

Within the meaning of the invention, an optical element is an element that acts on a beam for the purpose of modifying said beam. The action that modifies the beam may be a focusing of the beam, a scattering of the beam, a deflection of the beam, an alteration of the cross-sectional shape of the beam, a change in the optical path length of rays passing therethrough, etc. The action that modifies the beam may be implemented refractively, reflectively or diffractively.

In the ophthalmological optical observation apparatus according to the invention, the contactless fundus imaging system also fulfills a second function, specifically the function of a laser protection filter, in addition to its function of enabling the imaging of the fundus. As a result of the laser protection filter being formed by at least one coating of at least one optical element of the contactless fundus imaging system, it is not necessary to pivot an additional laser protection filter, i.e. an additional element with a transmission characteristic suitable for blocking the laser radiation, into the beam path of the ophthalmological optical imaging apparatus.

Moreover, the configuration of the ophthalmological optical observation apparatus according to the invention offers the additional advantage that during the treatment of the fundus by means of laser radiation the fundus is also observed using the contactless fundus imaging system, and so the laser protection filter is immediately also introduced into the beam path on account of the observation of the fundus, whereby the safety for the treating physician is increased during a treatment of the fundus. Moreover, there is no need to actuate a further actuating element for the purpose of pivoting-in the laser protection filter, increasing the user-friendliness of the ophthalmological optical observation apparatus.

Since the laser radiation generally has a very narrow bandwidth, the coating may have a transmission characteristic that blocks a very narrow spectral range. Typically, it is possible to use a transmission characteristic with a stop band that has a width of only 20 nm, in particular 10 nm, and that is centered about the centroid wavelength of the laser radiation, which may be at 532 nm, for example. Since the wavelength range removed from the spectrum, being 20 nm, in particular 10 nm, is very narrow, blocking the laser radiation has only a very small influence on the color representation attained by the optical observation apparatus. As a result, color distortions are so small that the coating that forms the laser protection filter does not noticeably impair the use of the ophthalmological optical observation apparatus away from a treatment of the fundus by means of laser radiation.

The transmission characteristic may be attained by a single coating that blocks only a narrow transmission range. However, it is alternatively also possible to realize the transmission characteristic by way of two coatings, which each have an edge in the transmission. Should one of the two coatings have a high transmission in a wavelength range below a first cut-off wavelength and a low transmission above the first cut-off wavelength, the second of the two coatings have a low transmission in a wavelength range below a second cut-off wavelength and a high transmission above the second cut-off wavelength and the first cut-off wavelength be below the second cut-off wavelength, a narrowband transmission range can be obtained using the two coatings. In this case, the two coatings may be applied to the same optical element or to different optical elements of the contactless fundus imaging system.

Should the contactless fundus imaging system comprise a focusing lens system, the at least one optical element may be part of the focusing lens system in particular. In general, the focusing lens system is arranged in the vicinity of the main objective of the optical observation apparatus, and so even laser radiation that has been scattered multiple times can be particularly reliably prevented from entering the observation beam path of the optical observation apparatus.

In general, an optical element of the contactless fundus imaging system comprises at least two optically effective surfaces. In an advantageous configuration of the ophthalmological optical observation apparatus according to the invention, the at least one coating is then present on that optical surface of the at least one optical element of the contactless fundus imaging system on which the incident light beams have the smallest angle of incidence on average. In this case, the average may for example be the arithmetic mean of the angles between the light rays of the beam paths incident on the optical surface and the surface normal at the respective point of incidence. However, a weighted mean or a root mean square also comes into question as a matter of principle. Especially if the at least one coating takes the form of an interference layer system, the effect of the laser protection filter deteriorates as the angle of incidence increases, and so a small angle of incidence is advantageous.

As optical elements, the focusing lens system may comprise at least one object-side lens and one image-side lens. In that case, the optical element provided with at least one coating preferably is the object-side lens of the focusing lens system. This lens has an object-side lens surface on which the coating has preferably been applied. In the focusing lens system, the object-side lens frequently contains the lens surface on which incident light beams have the smallest angles of incidence on average. In general, this surface is the object-side lens surface of the object-side lens.

According to a second aspect of the invention, a method is made available for providing an ophthalmological optical observation apparatus, which comprises a contactless fundus imaging system, with a laser protection filter with a transmission characteristic suitable during a treatment of the eye by means of laser radiation for blocking the laser radiation used during the treatment of the eye. In the method, at least one optical element of the contactless fundus imaging system is provided with at least one coating that realizes the transmission characteristic in order to form the laser protection filter.

As a result of at least one optical element of the contactless fundus imaging system being provided with the coating, the contactless fundus imaging system is also able to fulfill a second function, specifically the function of a laser protection filter, in addition to its function of enabling the imaging of the fundus. Moreover, this makes it possible to manage without an additional laser protection filter, i.e. an additional element with a transmission characteristic suitable for blocking the laser radiation and capable of being pivoted into the beam path, and so there is no need to actuate a further actuation element for the purpose of pivoting-in the laser protection filter, increasing the user-friendliness of the ophthalmological optical observation apparatus. Otherwise, reference is made to the advantages described in relation to the ophthalmological optical observation apparatus.

Should the contactless fundus imaging system comprise a focusing lens system, at least one optical element of the focusing lens system may be provided with the coating. In general, the focusing lens system is arranged in the vicinity of the main objective of the optical observation apparatus, and so even laser radiation that has been scattered multiple times can be particularly reliably prevented from entering the observation beam path of the optical observation apparatus.

Typically, the contactless fundus imaging system comprises at least one optical element comprising a plurality of optically effective surfaces. By preference, the optically effective surface on which incident light beams have the smallest angle of incidence on average is provided with the at least one coating. In this case, the average may for example be the arithmetic mean of the angles between the light rays of the beam paths incident on the optical surface and the surface normal at the respective point of incidence. However, a weighted mean or a root mean square also comes into question as a matter of principle. Especially if the at least one coating takes the form of an interference layer system, the effect of the laser protection filter deteriorates as the angle of incidence increases, and so a small angle of incidence is advantageous.

As optical elements, the focusing lens system of the contactless fundus imaging system may comprise at least one object-side lens and one image-side lens. In this case, it is advantageous if the object-side lens of the focusing lens system, preferably the object-side lens surface thereof, is provided with the at least one coating since the object-side lens frequently contains the lens surface on which incident light beams have the smallest angle of incidence on average. In general, this surface is the object-side lens surface of the object-side lens.

According to a third aspect of the present invention, a contactless fundus imaging system is made available, having at least one optical element and a laser protection filter with a transmission characteristic suitable during a treatment of the eye by laser radiation for blocking the laser radiation used. At least one optical element of the contactless fundus imaging system is provided with at least one coating that realizes the transmission characteristic and thus forms the laser protection filter.

Such a contactless fundus imaging system can be used advantageously in an ophthalmological optical observation apparatus that should find use for use in a treatment of the eye by means of laser radiation. Using the contactless fundus imaging system according to the invention, it is possible in the optical observation apparatus to manage without an additionally present laser protection filter capable of being pivoted-in, affecting the complexity of the apparatus and increasing user-friendliness. As regards the advantages and possible developments of the contactless fundus imaging system according to the invention, reference is made to the advantages and developments described in relation to the ophthalmological optical observation apparatus, from which the advantages and possible developments of the contactless fundus imaging system are immediately evident.

Further features, properties and advantages of the invention will become apparent from the following description of exemplary embodiments with reference to the accompanying figures.

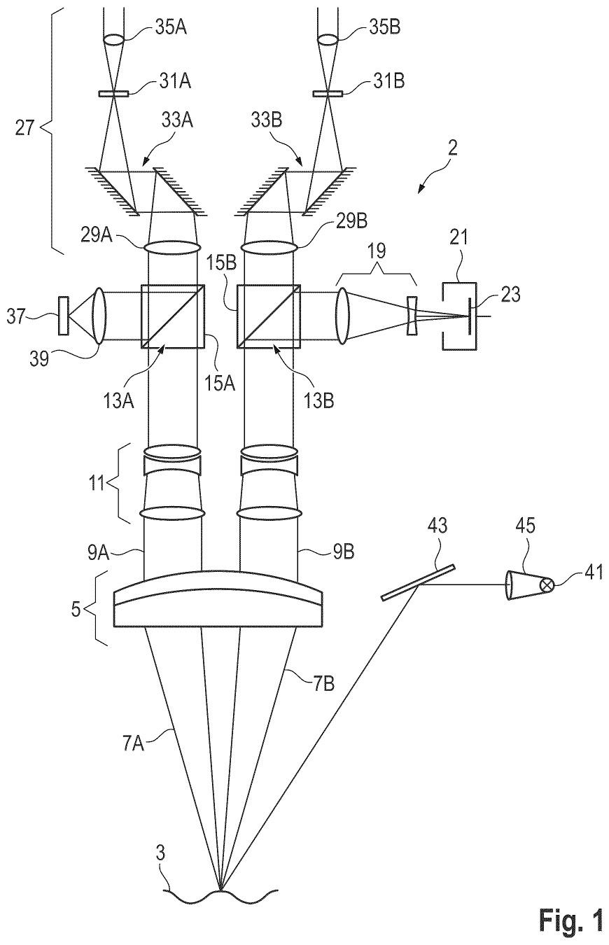

FIG. 1 shows a first example of a surgical microscope as an example of an ophthalmological optical observation apparatus.

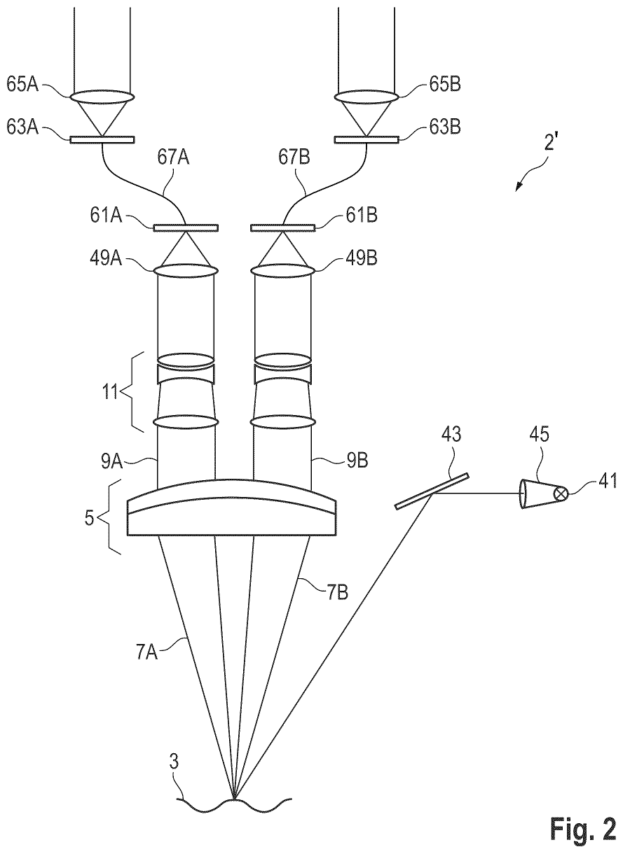

FIG. 2 shows a second example of a surgical microscope as an example of an ophthalmological optical observation apparatus.

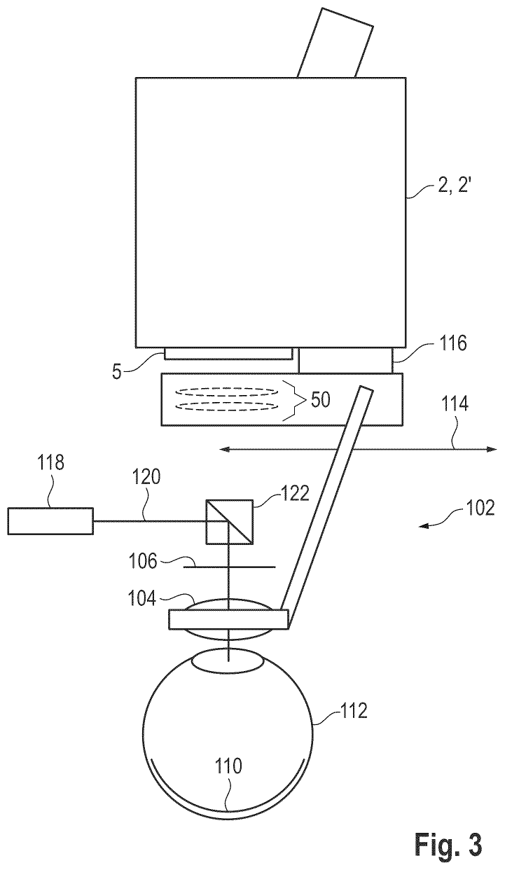

FIG. 3 shows an example of an ophthalmological optical observation apparatus taking the form of a surgical microscope and equipped with a contactless fundus imaging system.

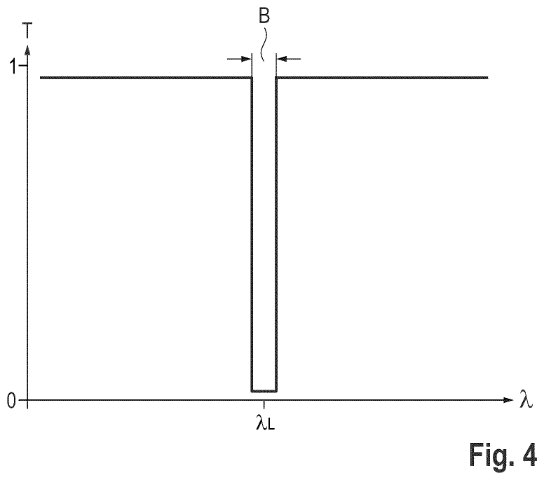

FIG. 4 shows a first example of a transmission characteristic of a laser protection filter.

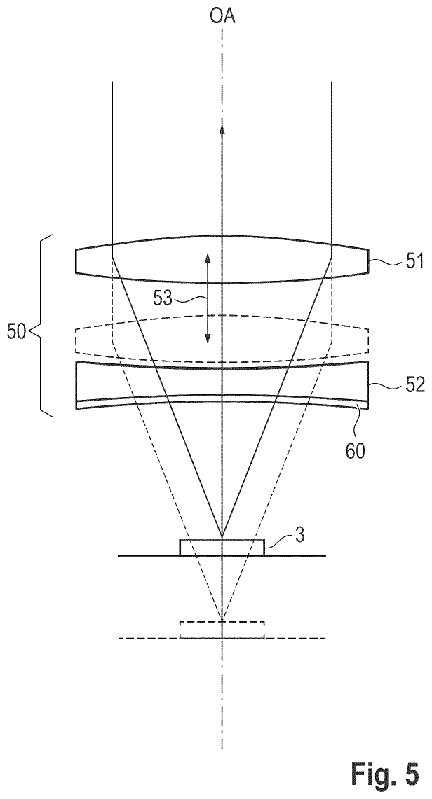

FIG. 5 shows a first example of a focusing lens system as may be used in the contactless fundus imaging system.

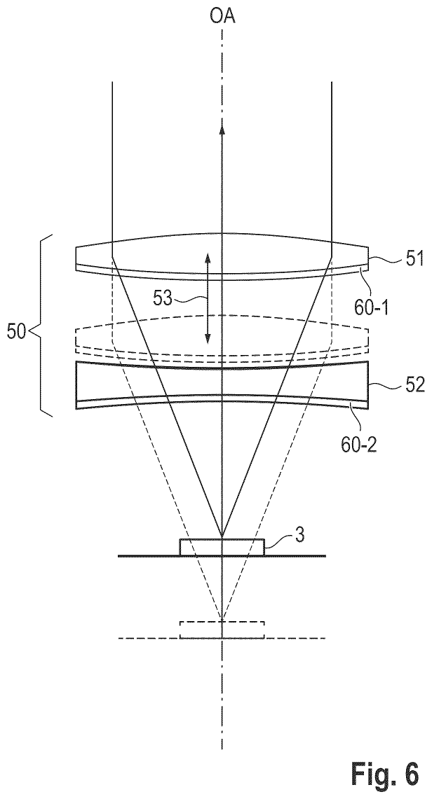

FIG. 6 shows a second example of a focusing lens system as may be used in the contactless fundus imaging system.

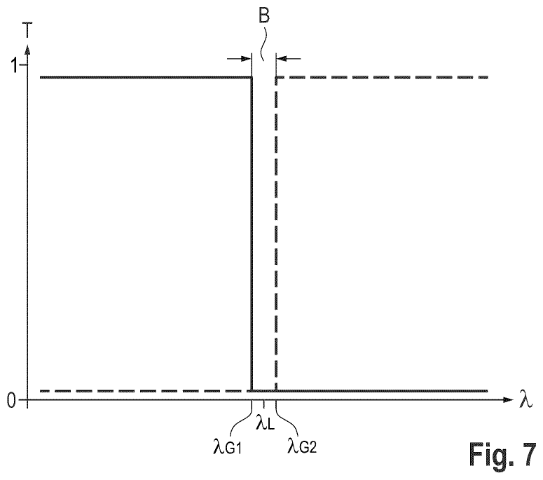

FIG. 7 shows an example of a transmission characteristic of a laser protection filter consisting of at least two coatings.

A surgical microscope 2 as an exemplary embodiment of an ophthalmological optical observation apparatus that can be used in eye surgery is described below with reference to FIG. 1. As essential component parts, the surgical microscope 2 shown in FIG. 1 comprises an objective 5 that faces an object field 3 and may be embodied as an achromatic or apochromatic objective in particular. In the present exemplary embodiment, the objective 5 consists of two partial lenses that are cemented to one another and together form an achromatic objective 5.

The object field 3 is arranged in the focal plane of the objective 5 such that it is imaged at infinity by the objective 5. In other words, a divergent beam 7A, 7B emanating from the object field 3 is converted into a parallel beam 9A, 9B during its passage through the objective 5. The beams 7A, 7B and 9A, 9B define beam paths of the surgical microscope, specifically stereoscopic partial beam paths.

A magnification changer 11 is arranged on the observer side of the objective 5 and may be embodied either as a zoom system for changing the magnification factor in a continuously variable manner or as what is known as a Galilean changer for changing the magnification factor in a stepwise manner. In a zoom system, constructed by way of example from a lens combination having three lenses, the two object-side lenses may be displaced in order to vary the magnification factor. In actual fact, however, the zoom system also may comprise more than three lenses, for example four or more lenses, in which case the outer lenses then may also be arranged in a fixed manner. In a Galilean changer, by contrast, there are a plurality of fixed lens combinations which represent different magnification factors and which can be introduced into the stereoscopic partial beam paths defined by the component beams 9A, 9B in alternation. Both a zoom system and a Galilean changer convert an object-side parallel beam into an observer-side parallel beam with a different beam diameter. In the present exemplary embodiment, the magnification changer 11 is already part of the binocular beam path of the surgical microscope 1, i.e. it has a dedicated lens combination for each stereoscopic partial beam path 9A, 9B of the surgical microscope 1. In the present exemplary embodiment, a magnification factor is set by means of the magnification changer 11 by way of a motor-driven actuator which, together with the magnification changer 11, is part of a magnification changing unit for setting the magnification factor.

The magnification changer 11 is adjoined on the observer side by an interface arrangement 13A, 13B, by means of which external equipment may be connected to the surgical microscope 2 and which comprises beam splitter prisms 15A, 15B in the present exemplary embodiment. However, other types of beam splitters may also be used in principle, for example partly transmissive mirrors. In the present exemplary embodiment, the interfaces 13A, 13B serve to output couple a beam from the stereoscopic partial beam path 9B of the surgical microscope 2 (beam splitter prism 15B) and to input couple a beam into the stereoscopic partial beam path 9A of the surgical microscope 2 (beam splitter prism 15A).

In the present exemplary embodiment, the beam splitter prism 15A in the stereoscopic partial beam path 9A serves to reflect information or data for an observer via the beam splitter prism 15A into the stereoscopic partial beam path 9A of the surgical microscope 2 with the aid of a display 37, for example a digital mirror device (DMD) or an LCD display, and an associated optical unit 39. A camera adapter 19 with a camera 21 fastened thereto, said camera being equipped with an electronic image sensor 23, for example with a CCD sensor or a CMOS sensor, is arranged at the interface 13B in the other stereoscopic partial beam path 9B. By means of the camera 21, it is possible to record an electronic image, and in particular a digital image, of the tissue region 3, for instance for documentation purposes or for displaying an image of the object field 3 on a monitor.

A binocular tube 27 adjoins the interface 13 on the observer side. It has two tube objectives 29A, 29B, which focus the respective parallel beam 9A, 9B on an intermediate image plane 31, i.e. image the observation object 3 onto the respective intermediate image plane 31A, 31B. Finally, the intermediate images situated in the intermediate image planes 31A, 31B are imaged in turn at infinity by eyepiece lenses 35A, 35B, and so a viewer can view the intermediate image with a relaxed eye. Moreover, an increase in the distance between the two component beams 9A, 9B is implemented in the binocular tube by means of a mirror system or by means of prisms 33A, 33B in order to adapt said distance to the interocular distance of the viewer. In addition, image erection is carried out by the mirror system or the prisms 33A, 33B.

The surgical microscope 2 is also equipped with illumination equipment, by means of which the object field 3 can be illuminated with broadband illumination light. To this end, the illumination equipment in the present exemplary embodiment has a white-light source 41, for example a halogen lamp or a gas discharge lamp. The light emanating from the white-light source 41 is directed via a deflection mirror 43 or a deflection prism in the direction of the object field 3 in order to illuminate the latter. Furthermore, an illumination optics unit 45 is present in the illumination equipment and ensures uniform illumination of the entire observed object field 3.

Reference is made to the fact that the illumination beam path depicted in FIG. 1 is highly schematic and does not necessarily reproduce the actual course of the illumination beam path. In principle, the illumination beam path may take the form of what is known as oblique illumination, which comes closest to the schematic illustration in FIG. 1. In such oblique illumination, the beam path extends at a relatively large angle (6° or more) with respect to the optical axis of the objective 5 and, as illustrated in FIG. 1, may extend completely outside the objective. In an alternative, however, there is also the option of allowing the illumination beam path of the oblique illumination to extend through a marginal region of the objective 5. A further possibility for the arrangement of the illumination beam path is so-called 0° illumination, in which the illumination beam path extends through the objective 5 and is input coupled into the objective between the two partial beam paths 9A, 9B, along the optical axis of the objective 5 in the direction of the object field 3. Finally, it is also possible to design the illumination beam path as what is known as coaxial illumination, in which a first illumination partial beam path and a second illumination partial beam path are present. The partial beam paths are input coupled into the surgical microscope in a manner parallel to the optical axes of the observation partial beam paths 9A, 9B by way of one or more beam splitters such that the illumination extends coaxially in relation to the two observation partial beam paths.

A digital surgical microscope as an exemplary embodiment of an ophthalmological optical observation apparatus that can be used in eye surgery is described below with reference to FIG. 2. In the digital surgical microscope 2′, the main objective 5 with the coating 6 for filtering out the laser radiation 47, the magnification changer 11, which merely represents an option in the digital surgical microscope 2′ and hence need not necessarily be present, and the illumination system 41, 43, 45 do not differ from the surgical microscope 2 with an optical viewing unit, depicted in FIG. 1. The difference lies in the fact that the surgical microscope 2′ shown in FIG. 2 does not comprise an optical binocular tube. Instead of the tube objectives 29A, 29B from FIG. 1, the surgical microscope 2′ from FIG. 2 comprises focusing lenses 49A, 49B, with which the binocular observation beam paths 9A, 9B are imaged onto digital image sensors 61A, 61B. In this case, the digital image sensors 61A, 61B may be CCD sensors or CMOS sensors, for example. The images recorded by the image sensors 61A, 61B are transmitted to digital displays 63A, 63B, which may be embodied as LED displays, as LCD displays, or as displays based on organic light-emitting diodes (OLEDs). Like in the present example, eyepiece lenses 65A, 65B may be assigned to the displays 63A, 63B, by means of which the images displayed on the displays 63A, 63B are imaged at infinity such that an observer can observe said images with relaxed eyes. The displays 63A, 63B and the eyepiece lenses 65A, 65B may be part of a digital binocular tube; however, they may also be part of a head-mounted display (HMD) such as for instance a pair of smartglasses. Even though FIG. 2 shows a transmission of the images recorded by the image sensors 61A, 61B to the displays 63A, 63B of a digital binocular tube by means of cables 67A, 67B, the images may also be transmitted wirelessly to the displays 63A, 63B, especially when the displays 63A, 63B are part of a display to be worn on the head. Moreover, there is the option of representing the recorded images as stereoscopic images on a large monitor that is observed by staff in the operating theater using suitable 3-D glasses. For the purpose of differentiating the partial stereoscopic images, the latter may be represented e.g. using different polarizations of the light emitted by the monitor during the representation of the stereoscopic images on the monitor. The 3-D glasses then contain switchable polarizers that are switched synchronously with the representation of the partial images on the monitor.

Use is made of a contactless fundus imaging system if the surgical microscope 2 depicted in FIG. 1 or the surgical microscope 2′ depicted in FIG. 2 should find use in posterior segment surgery for example. A surgical microscope 2, 2′ with a contactless fundus imaging system 102 is depicted in FIG. 3. The contactless fundus imaging system 102 is needed because the fundus 110 of an eye 112 cannot be readily observed using the surgical microscope 2, 2′. Thus, for the purpose of observing the fundus 110, the contactless fundus imaging system 102 comprises what is known as an ophthalmic loupe 104, which creates an aerial image of the fundus 110 in an intermediate image plane 106 that is then observed using the surgical microscope 2, 2′. Since the surgical microscope 2, 2′ comprises a fixed-focal-length objective 5, the fundus imaging system 102 moreover comprises a focusing lens system 50 that serves to shorten the focal distance of the surgical microscope 2, 2′ to such an extent that it is possible to observe the aerial image in the intermediate image plane 106. The focusing lens system 50 will be described in detail hereinafter with reference to FIGS. 6 and 7.

To observe the fundus 110 of an eye 112, the contactless fundus imaging system 102 can be pushed into the observation beam path of the surgical microscope 2, 2′ by means of a sliding mechanism 116. Should the fundus 110 no longer be observed, the contactless fundus imaging system may be pushed out of the beam path again. Pushing the contactless fundus imaging system 102 in and out is symbolized by the double-headed arrow 114 in FIG. 3.

In particular, the fundus 110 is also observed when there should be a laser treatment of the fundus 110. In this context, a laser beam 120 emitted by a laser 118 is for example deflected by way of a beam splitter 122 in the direction of the fundus 110 of the eye 112. By contrast, light emanating from the eye in the direction of the surgical microscope 2, 2′ is passed by the beam splitter 122 without deflection. Hence, reflected or scattered laser light may also reach into the surgical microscope 2, 2′. This may endanger the eyes of the treating surgeon or lead to the actual image content being swamped by laser light in images recorded by electronic image sensors.

The introduction of a laser protection filter into the observation beam path of the surgical microscope 2, 2′ is a measure that can prevent the eyes of the treating surgeon from being endangered or the image content being swamped in images recorded by electronic image sensors. In general, such a laser protection filter has a transmission characteristic with a narrow stop band that blocks only a narrow range around the centroid wavelength of the laser light 120.

A transmission characteristic for a laser protection filter is shown schematically in FIG. 4. The transmission characteristic has a very low transmission T close to 0 in a narrow spectral range around the centroid wavelength λL of the laser radiation 120, whereas it has a high transmission T close to 1 in all remaining spectral ranges. Hence the laser protection filter acts as a band-stop filter which blocks the narrow spectral range around the centroid wavelength λL of the laser radiation 120. In this context, the width of the stop band B of the laser protection filter is typically a few nanometers, for example no more than 20 nm, preferably no more than 10 nm, with the centroid wavelength λL of the laser radiation 120 forming the center of the stop band B. In the present exemplary embodiment, the centroid wavelength λL of the laser radiation is 532 nm. A transmission characteristic as depicted in FIG. 4 may be realized using a spectral filter that absorbs or reflects the narrow spectral range around the centroid wavelength λL of the laser radiation. However, the transmission characteristic can be realized particularly advantageously with the aid of an interference layer system. In such an interference layer system, filtering is not based on absorption or scattering but on reflection, created by interference, in the narrow spectral range around the centroid wavelength λL of the laser radiation 120. In the present exemplary embodiment, the laser protection filter is realized by a coating in the focusing lens system 50 of the contactless fundus imaging system 102. A first example of a focusing lens system 50 of the contactless fundus imaging system 102 with a coating 60 that acts as a laser protection filter is depicted schematically in FIG. 5.

The focusing lens system 50 in the first example comprises a positive member 51, i.e. an optical element with positive refractive power, depicted schematically as a convex lens in FIG. 5. Moreover, the focusing lens system 50 comprises a negative member 52, i.e. an optical element with negative refractive power, depicted schematically as a concave lens in FIG. 5. The negative member 52 is situated between the positive member 51 and the object field 3, 3′. In the depicted focusing lens system 50, the negative member 52 has a fixed arrangement, whereas, as indicated by the double-headed arrow 53, the positive member 51 is arranged to be displaceable along the optical axis OA. Should the positive member 51 in FIG. 5 be displaced from the position depicted using dashed lines to the position depicted using solid lines, the back focus of the focusing lens system 50 is shortened such that the focal distance is reduced and hence the object field 3, 3′ may be located closer to the focusing lens system 50, whereby it is possible to observe the aerial image in the intermediate image plane 106.

In the present exemplary embodiment, the focusing lens system 50 has four optically effective surfaces, specifically the surfaces of the positive member 51 and negative member 52 facing the object field 3 and the surfaces of the positive member 51 and negative member 52 facing away from the object field 3. In the present exemplary embodiment, the surface of the negative member 52 facing the object field 3 is provided with a coating 60 that has the transmission characteristic shown in FIG. 4. Hence, the lens 52 of the focusing lens system simultaneously forms the laser protection filter.

A second example of a focusing lens system of the contactless fundus imaging system is depicted in FIG. 6. Apart from the embodiment of the coating, this focusing lens system does not differ from the focusing lens system shown in FIG. 5. The lenses of the focusing lens system from FIG. 6 and the focusing lens system itself are therefore denoted by the same reference signs as the lenses 51, 52 and the focusing lens system 50 from FIG. 5, and in order to avoid repetition the function of the lenses 51, 52 of the focusing lens system 50 is not explained again.

However, in contrast to the focusing lens system from FIG. 5, it is not only the object-side lens surface of the lens 52 but also the object-side lens surface of the lens 51 that is provided with a coating in the focusing lens system shown in FIG. 6. The coatings 60-1 and 60-2 form the laser protection filter together in the present example. These coatings 60-1, 60-2 each act as an edge filter, wherein the first coating 60-1 has a first cut-off wavelength λ_{G1} and the second coating 60-2 has a second cut-off wavelength λ_{G2}, with the cut-off wavelength λ_{G1} being shorter than the second cut-off wavelength λ_{G2}, as shown in FIG. 7. Hence, the coatings 60-1, 60-2 together lead to a transmission characteristic with a narrow stop band B centered around the centroid wavelength λL of the laser radiation 120. In this case, it is irrelevant which of the two coatings 60-1, 60-2 is the coating with the first cut-off wavelength λ_{G1} and which is the coating with the second cut-off wavelength λ_{G2}.

Even though the positive member 51 has a displaceable configuration in FIGS. 5 and 6, it is also possible, in principle, to arrange the negative member 52 to be movable along the optical axis OA instead of the positive member 51. However, the negative member 52 often forms the last lens element of the focusing lens system 50. A stationary negative member 52 therefore offers the advantage of making it easier to seal the interior of the focusing lens system from external influences. Furthermore, it is noted that even though the positive member 51 and the negative member 52 in FIG. 5 are only illustrated as individual lens elements, each of these members may also be realized in the form of a lens group or a cemented element instead of in the form of an individual lens, for example to design the focusing lens system to be achromatic or apochromatic.

The present invention has been described in detail on the basis of exemplary embodiments for explanatory purposes. However, a person skilled in the art recognizes that there can be deviations from the exemplary embodiments within the scope of the present invention, as claimed in the attached claims. For example, it is possible to arrange the coating in the region of a cemented surface if the focusing lens system comprises at least one cemented member. Moreover, it is possible to design the contactless fundus imaging system to be capable not of being pushed in by means of a linear movement but of being pivoted in by means of a pivoting movement. Moreover, it is possible that the contactless fundus imaging system comprises at least two ophthalmic loupes that are arranged in a turret mechanism and that can find use in alternation. Therefore, the present invention is not intended to be limited by the exemplary embodiments but rather only by the appended claims.

### LIST OF REFERENCE SIGNS

2, 2° Surgical microscope
3 Object field
5 Objective
5-1 Lens
5-2 Lens
7A,B Divergent beam
9A,B Stereoscopic partial beam path
11 Magnification changer
13A,B Interface arrangement
15A,B Beam splitter prism
19 Camera adapter
21 Camera
23 Image sensor
37 Display
39 Optics unit
41 White-light source
43 Deflection mirror
45 Illumination optics unit
47 Laser radiation
49A,B Focusing lens
50 Focusing lens system
51 Positive member
52 Negative member
53 Displacement path
60 Coating
60-1 Coating
60-2 Coating
61A,B Image sensor
63A,B Display
65A,B Eyepiece lens
67A,B Cable
102 Fundus imaging system
104 Ophthalmic loupe
106 Intermediate image plane
110 Fundus
112 Eye
114 Double-headed arrow
116 Sliding mechanism
118 Laser
120 Laser radiation
122 Beam splitter
B Stop band
H Main observer beam path
M Co-observer beam path
OA Optical axis
T Transmission

1. An ophthalmological optical observation apparatus for use during a treatment of the eye by laser radiation, comprising:
a contactless fundus imaging system having at least one optical element and
a laser protection filter with a transmission characteristic suitable for blocking the laser radiation used during the treatment of the eye, wherein at least one optical element of the contactless fundus imaging system is provided with at least one coating that realizes the transmission characteristic and thus forms the laser protection filter.

2. The optical observation apparatus as claimed in claim 1, wherein an objective and an intermediate image plane located between the objective and an eye to be treated, wherein the contactless fundus imaging system is arranged between the objective and the intermediate image plane, and the at least one optical element of the fundus imaging system is configured to enable an observation of an aerial image of the fundus, created in the intermediate image plane, by the ophthalmological optical observation apparatus.

3. The optical observation apparatus as claimed in claim 1, wherein the contactless fundus imaging system comprises a focusing lens system, and the optical element is part of the focusing lens system.

4. The optical observation apparatus as claimed in claim 1, wherein the contactless fundus imaging system comprises a focusing lens system, and the optical element is part of the focusing lens system, wherein the contactless fundus imaging system has at least one optical element that comprises a plurality of optically effective surfaces, and the at least one coating is present on that optical surface on which incident light beams have the smallest angle of incidence on average, wherein the focusing lens system comprises at least one object-side lens and one image-side lens as optical elements, and the optical element provided with at least one coating is the object-side lens of the focusing lens system.

5. The optical observation apparatus as claimed in claim 4, wherein the object-side lens comprises an object-side lens surface, and the coating is arranged on the object-side lens surface.

6. A method for providing an ophthalmological optical observation apparatus, which comprises a contactless fundus imaging system, with a laser protection filter with a transmission characteristic suitable during a treatment of the eye by means of laser radiation (120) for blocking the laser radiation used during the treatment of the eye,
wherein at least one optical element of the contactless fundus imaging system is provided with at least one coating that realizes the transmission characteristic in order to form the laser protection filter.

7. The method as claimed in claim 6, wherein the contactless fundus imaging system has at least one optical element that comprises a plurality of optically effective surfaces, and the optical surface on which incident light beams have the smallest angle of incidence on average is provided with the at least one coating.

8. The method as claimed in claim 6, wherein the contactless fundus imaging system comprises a focusing lens system, and the optical element that is provided with at least one coating is an optical element of the focusing lens system.

9. The method as claimed in claim 8, wherein the focusing lens system comprises at least one object-side lens and one image-side lens as optical elements, and the object-side lens of the focusing lens system is provided with the at least one coating.

10. The method as claimed in claim 9, wherein the object-side lens of the focusing lens system comprises an object-side lens surface on which the coating is arranged.

11. A contactless fundus imaging system having at least one optical element and a laser protection filter with a transmission characteristic suitable during a treatment of the eye by laser radiation for blocking the laser radiation used,
wherein at least one optical element of the contactless fundus imaging system is provided with at least one coating that realizes the transmission characteristic and thus forms the laser protection filter.

12. The optical observation apparatus as claimed in claim 1, wherein the contactless fundus imaging system has at least one optical element that comprises a plurality of optically effective surfaces, and the at least one coating is present on that optical surface on which incident light beams have the smallest angle of incidence on average.

13. The method as claimed in claim 6, wherein the ophthalmological optical observation apparatus contains an objective and an intermediate image plane is present between the objective and an eye to be treated, wherein the contactless fundus imaging system is arranged between the objective and the intermediate image plane, and the at least one optical element of the fundus imaging system enables an observation of an aerial image of the fundus, created in the intermediate image plane, by the ophthalmological optical observation apparatus.
